(19) United States
(12) Patent Application Publication
(10) Pub. No.: US 2019/0290199 A1
(43) Pub. Date: Sep. 26, 2019

(54) SYSTEM FOR GENERATING, EMITTING AND INTERPRETING A COMPOSITE STREAM, AND ASSOCIATED METHOD

(71) Applicant: SYNAPSYS, Marseille (FR)

(72) Inventor: Stephane CURCIO, Marseille (FR)

(73) Assignee: SYNAPSYS, Marseille (FR)

(21) Appl. No.: 15/776,344

(22) PCT Filed: Nov. 15, 2016
(86) PCT No.: PCT/FR2016/052961

§ 371 Date: May 15, 2018

Related U.S. Application Data
(60) Provisional application No. 62/255,821, filed on Nov. 16, 2015.

(30) Foreign Application Priority Data
May 31, 2016 (FR) 1654875

(51) Int. Cl. A61B 5/00; A61B 3/113; H04N 19/167; G06K 9/00

(52) U.S. Cl. CPC A61B 5/4863; G06K 9/00604; H04N 19/167; A61B 3/113

(57) ABSTRACT

System for generating, emitting and interpreting a composite stream and associated method. The invention relates to a system making it possible to generate, transmit, interpret and exploit a composite data stream. The invention relates more precisely to a method for emitting a composite data stream advantageously through a wireless communication network, and a method for interpreting a composite data stream. Said methods are respectively implemented by the processing unit of an electronic object and by the processing unit of an electronic device.

The invention relates to a method for emitting a composite data stream, said data being made up of data of interest and environmental data respectively characterizing the digital production of information of interest and environmental information, comprised in one or several pieces of study information previously captured by capture means. The invention further relates to a method for interpreting and exploiting such a composite data stream and a system making it possible to carry out said methods.

More particularly, but non-limitingly, the invention relates to the sending of a video stream in a videonystagmoscopy system used to observe ocular movements in humans or animals, or more generally a study subject, and to look for any nystagmus. A nystagmus is an involuntary and jerky movement of the eyeball caused by a disruption in the muscles of the eye. The observation of a nystagmus may, as one non-limiting example, make it possible to determine a dysfunction of the inner ear in a patient that may cause vertigo.

Vertigo is an incorrect sensation of movement of the body relative to the surrounding area generally reflecting a dysfunction or imbalance between the two vestibular apparatuses of the inner ear of a human or animal. Each vestibular apparatus is made up of several sensors, such as semicircular channels and otolithic organs, the walls of which are covered with ciliated sensory cells bathing in endolymphatic liquid. The semicircular channels detect the amplitude of the angular rotation of the head, while the otolithic organs detect vertical and/or horizontal linear accelerations of the head as well as the incline of the head relative to the axis of gravity. During a movement caused, for example but not limited to, during a rotation of the head, the ciliated cells move and send the information related to the rotation to the nervous system through the vestibular nerve. The interpretation by the patient's nervous system of such information causes movements of the eyeball in order to guarantee stability of the patient's gaze and posture.

When a dysfunction of the inner ear occurs, and more particularly of the vestibular apparatus, said stabilizations are not done correctly, causing a nystagmus, as well as the feeling of vertigo.

A videonystagmoscopy system generally includes a helmet, a mask or glasses including one or several cameras and an electronic device, for example, but not limited to, a tablet or personal computer. During examination of the inner ear, commonly called vestibular examination, the patient's eye is plunged into darkness, thus preventing its inhibition relative to fixation, which could alter the result of the exam. The movements of the eyeball are then caused only by the patient's nervous system following the information received from the sensors of said patient's inner ear. The camera, generally of the infrared type, captures a series of images in order to create a video stream. The latter is sent to the electronic device, responsible for outputting images or graphics via a man-machine output interface, for example, but not limited to, a computer monitor or a printer. The practitioner can then analyze the output and make a diagnosis in light of this tool.

The observation of a nystagmus requires a high resolution of the image. Indeed, the jerky horizontal and/or vertical movements of the eyeball and/or torsion thereof are low-amplitude events. Furthermore, said events can be very short. The video stream must then be sent between 25 Hz and 200 Hz for images with 256 gray levels and able to reach up to a size of 640×480 pixels. The stream thus generated between the infrared camera and the electronic device conveys a large quantity of information expressed in the form of bit frames, thus occupying a large bandwidth. In order to make the output of the stream ergonomic and usable, it is necessary to offer a transmission of data packets with a low lag or delay. It is therefore crucial to calibrate the bandwidth of the communication network and the computing power of the electronic device, to reorganize the incoming data and decode it.

To send such a volume of data without difficulties, videonystagmoscopy systems generally include a mask and a device communicating by wired link, for example using a connector cable of the USB (Universal Serial Bus) type. Such systems have the drawback of hindering the practitioner and the patient during the exam. Indeed, during an exam of the vestibular system, the practitioner must cause the patient's head to turn from left to right, have the patient shake his head and/or turn the chair in which the patient is sitting at different speeds, in order to observe any distortions of the eyes and determine the malfunctioning organ causing the vertigo. The drawback of this system thus lies primarily in said cable connecting the camera positioned in front of the patient's eyes to the practitioner's computer. This cable may in fact become wound around the patient during the exam. Furthermore, the USB standard may have an insufficient bandwidth, thereby causing delays when receiving data packets or the loss of data packets.

To offset this drawback related to the presence of the cable, several videonystagmoscopy systems propose sending the data captured by the camera to the practitioner's computer by relying on dedicated or specialized radio-analog technologies. Such systems, however, have clipping and/or static problems based on the orientation of the antennas, during the movement of the patient during the exam, and are costly to acquire and maintain.

Other solutions have sought to rely on proximity communication protocols, for example of the Wi-Fi type. However, these solutions are ineffective because such wireless networks have an insufficient bandwidth relative to the needs of a videonystagmoscopy system, the video stream not being output correctly.

The invention makes it possible to respond particularly effectively to all or some of the drawbacks raised by the aforementioned solutions. To that end, the invention proposes to reduce the volume of data to be sent by performing processing operations upstream from the sending. Indeed, the invention provides for delegating, to a microcontroller previously installed in videonystagmoscopy glasses and cooperating with the camera, local processing operations upstream from the sending, in order to reduce the volume of data exchanged with an electronic device to just the information useful for the medical use of the product. Indeed, during a vestibular exam, it suffices to observe the iris and pupil of the patient's eye to diagnose a potential dysfunction of the inner ear, the rest of the image being of less interest, or at least not requiring the same precision. However, the observation of the rest of the eyeball, called surrounding area, provides the practitioner with visual comfort and remains desired by healthcare professionals. The invention then provides, first, for compressing and encoding the data of interest Di related to the zone of interest Zi according to a first encoding function F1 and the environmental data Dn related to the environmental zone Zn according to a second encoding function F2, said first encoding function having a data loss rate related to the compression of the data that is zero or low relative to said second encoding function F2. The data of interest Di and the environmental data Dn thus encoded will respectively be called encoded data of interest Di′ and encoded environmental data Dn′.

An encoding function F1, F2 refers to any transcription of data from a first format to a second format, able to include a step for compression of the data. Such a function F1, F2 may, as a non-limiting example, consist of transcribing the digital representation R(t) of physical properties captured by a matricial sensor of a camera encoded according to the ASCII (American Standard Code for Information Interchange) standard, a JPEG (Joint Photographic Expert Group) standard. Such functions may have reversible or irreversible compression functionalities. A reversible compression is a processing operation guaranteeing the integrity of the encoded data relative to the original data, i.e., after decompression, the encoded data and the original data are identical. As one non-limiting example, a transcription according to the RLE (Run Length Encoding) standard is an encoding using reversible compression. On the contrary, an irreversible compression is a processing operation that reduces the number of data to be encoded, for example by encoding only part of the data, as one non-limiting example every other datum, or by performing a prior processing operation to determine the mean of an adjacent data lot and by encoding said mean. As a non-limiting example, a transcription using the JPEG (Joint Photographic Expert Group) standard is encoding using irreversible compression.

Such a processing operation for the study data upstream from sending thereof makes it possible to preserve the bandwidth of the communication networks. The study data thus adapted can then be conveyed according to standard proximity communication protocols, for example but not limited to the Wi-Fi or Bluetooth type. It then becomes possible to use standard communication equipment, thereby reducing the hardware costs of the system while preserving the ergonomics and relevance of the system.

Furthermore, such a reduction in the volume of study data to be transmitted, said study data being generated from a determined sensor, for example a matricial image sensor, makes it possible, for a bandwidth of a given communication network, to convey, jointly with said study data, other study data produced by one or several other sensors, for example an accelerometer, a gyroscope and/or a second matricial image sensor. This study data stream can thus be considerably enriched.

To that end, the invention first relates to a method for interpreting a composite data stream implemented by a processing unit of an electronic device cooperating with at least one communicating electronic object including capture means, said device including, aside from said processing unit, communication means providing a communication mode with said electronic object through a communication network. The data stream includes study data previously encoded by the electronic object, said study data including data of interest encoded according to a first encoding function and environmental data encoded according to a second encoding function, said study data being produced beforehand by said capture means of said electronic object. To interpret such a composite data stream, the method includes:
a step for receiving, via the communication means, a message including said encoded study data;
a step for decoding said encoded study data, said step consisting of decoding said encoded data of interest and environmental data respectively according to first and second determined decoding functions;
a step for jointly exploiting said data of interest and environmental data thus decoded.

To be able to produce output data from decoded data of interest and environmental data, said message may further include a descriptor translating a coordinate system shared by the encoded data of interest and environmental data. The step for jointly exploiting said decoded data of interest and environmental data may then consist of producing output data from decoded data of interest and environmental data and said shared coordinate system.

To command the encoding functions implemented by the electronic object, the method may include a step, prior to the step for receiving a message including encoded data of interest and environmental data, for generating and triggering the sending by the communication means to the electronic object of a command request able to be interpreted by said electronic object including a first encoding parameter and a second encoding parameter, said first and second parameters being different.

To command the discrimination functions implemented by the electronic object, the method may include a step, prior to the step for receiving a message including encoded data of interest and environmental data, for generating and triggering the sending by the communication means to the electronic object of a command request able to be interpreted by said electronic object including a zone of interest parameter designating data of interest within study data produced by the capture means of the electronic object, said zone of interest parameter being able to be interpreted by the electronic object to discriminate the data of interest from the environmental data previously produced by said object.

To allow the output of the received composite data stream, the electronic device may include an output interface cooperating with the processing unit of said device. The step for jointly exploiting said decoded data of interest and environmental data may consist of producing output data. The method may then include a step after said step for jointly exploiting said decoded data of interest and environmental data, to trigger the output of said output data via the output interface.

According to a new object, the invention relates to a computer program product including program instructions which, when recorded beforehand in a program memory of an electronic device further including the program memory, a processing unit, and communication means providing a determined communication mode, said program memory and said communication means cooperating with said processing unit, cause the implementation of a method for interpreting a composite data stream, according to the invention.

According to a third object, the invention provides an electronic device including a processing unit, a program memory, a data memory, communication means cooperating with said processing unit, said device being characterized in that it includes, in the program memory, instructions for a computer program product according to the invention.

According to a fourth object, the invention provides a method for sending a composite data stream implemented by a processing unit of a communicating electronic object cooperating with at least one electronic device, said electronic object further including said processing unit of the capture means and communication means providing a communication mode with said electronic device through a communication network. To send such a composite data stream, said method includes:
a step for triggering the production of study data by said capture means of said electronic object, said study data consisting of a digital representation of a measured physical property;
a step for discriminating, in the study data, data of interest and environmental data according to a discrimination function;
a step for encoding the data of interest according to a first encoding function and environmental data according to a second encoding function;
a step for developing and triggering the sending, by the communication means to the electronic device, of a message including all or some of said encoded data of interest and/or environmental data.

In order to dynamically modify the parameters for implementing the first and second encoding functions, the method may include a step before the step for encoding the data of interest according to a first encoding function and the environmental data according to a second encoding function, for receiving, via the communication means, a command request sent from the electronic device including a first encoding parameter and a second encoding parameter, and extracting said parameters, said first and second encoding functions respectively being implemented according to the content of the first and second encoding parameters.

In order to dynamically modify the parameters for implementing said discrimination function, the method may include a step prior to the step for discriminating, in the study data, the data of interest and the environmental data according to a discrimination function, to receive, via the communication means, a command request sent from the electronic device including a zone of interest parameter designating data of interest and to extract said parameter, said discrimination function being implemented according to the content of said zone of interest parameter.

To modify the parameters for implementing the discrimination function, the electronic object may further include storage means including a zone of interest parameter designating data of interest from among the study data. Said method may then include a step prior to the step for discriminating, in the study data, data of interest and environmental data, to extract, from said storage means, said zone of interest parameter designating data of interest and carrying out a discrimination function according to the content of said parameter.

To modify the parameters for implementing encoding functions, the electronic object may further include storage means including first and second encoding parameters. The step for encoding the data of interest according to a first encoding function and the environmental data according to a second encoding function may then consist of extracting the content of said encoding parameters, said first and second encoding functions respectively being carried out according to said content of the first and second encoding parameters.

According to the invention, the capture means of the electronic object may include a matricial image sensor, the study data produced by said sensor being the digital representation of the scene captured by said matricial sensor. As a preferred example application, the study data may consist of the digital representation of an eye, and the data of interest may be the digital representation of the iris and the pupil of said eye.

According to a fifth object, the invention relates to a computer program product including program instructions which, when recorded beforehand in a program memory of a communicating electronic object further including said program memory, a processing unit, communication means providing a determined communication mode, and capture means, said program memory, said communication means, and said capture means cooperating with said processing unit, cause the implementation of a method for sending a composite data stream, according to the invention.

According to a sixth object, the invention relates to a communicating electronic object including a processing unit, a program memory, a data memory, communication means, capture means cooperating with said processing unit, said object being characterized in that it includes, in the program memory, instructions for a computer program product, according to the invention.

To exploit a data set related to a same study subject, the capture means may include a first capture means and a second capture means to respectively produce first and second study data. The processing unit may then carry out a method for sending a composite data stream, according to the invention, for each of said first and second capture means.

According to a seventh object, the invention relates to a system including an electronic device according to the invention, and to at least one communicating electronic object according to the invention.

According to one preferred example application, said system may consist of a videonystagmoscopy system, for which the electronic device consists of a personal computer and the electronic object consists of a videonystagmoscopy mask including at least one matricial image sensor.

According to an eighth object, the invention relates to a data processing method including:
a step for triggering a production of study data by capture means of a communicating electronic object according to the invention, said step being carried out by a processing unit of said object;
a step for discriminating, in the study data, data of interest and environmental data via said electronic object;
a step for encoding, via said object, the data of interest according to a first encoding function and the environmental data according to a second encoding function;
a step for generating, via said object, a message including said encoded data of interest and said encoded environmental data and triggering the sending to an electronic device according to the invention;
a step for receiving, via the device, said message and decoding it;
a step for jointly exploiting said decoded data of interest and environmental data via the device.

To make the encoding functions and the discrimination functions dynamic, the data processing method may include:
a step for developing and triggering the sending, through a communication network to the communicating electronic object, a command request including encoding parameters and a zone of interest parameter implemented by the processing unit of an electronic device;
a step for receiving, via the communicating electronic object, said command request and extracting said parameter;
the discriminating step being carried out according to the contents of the zone of interest parameter previously extracted;
a step for respectively encoding the data of interest and the environmental data being carried out by said object according to the content of said encoding parameters previously extracted.

Other features and advantages will appear more clearly upon reading the following description, relative to one example embodiment provided for information, and upon examining the accompanying figures, among which:

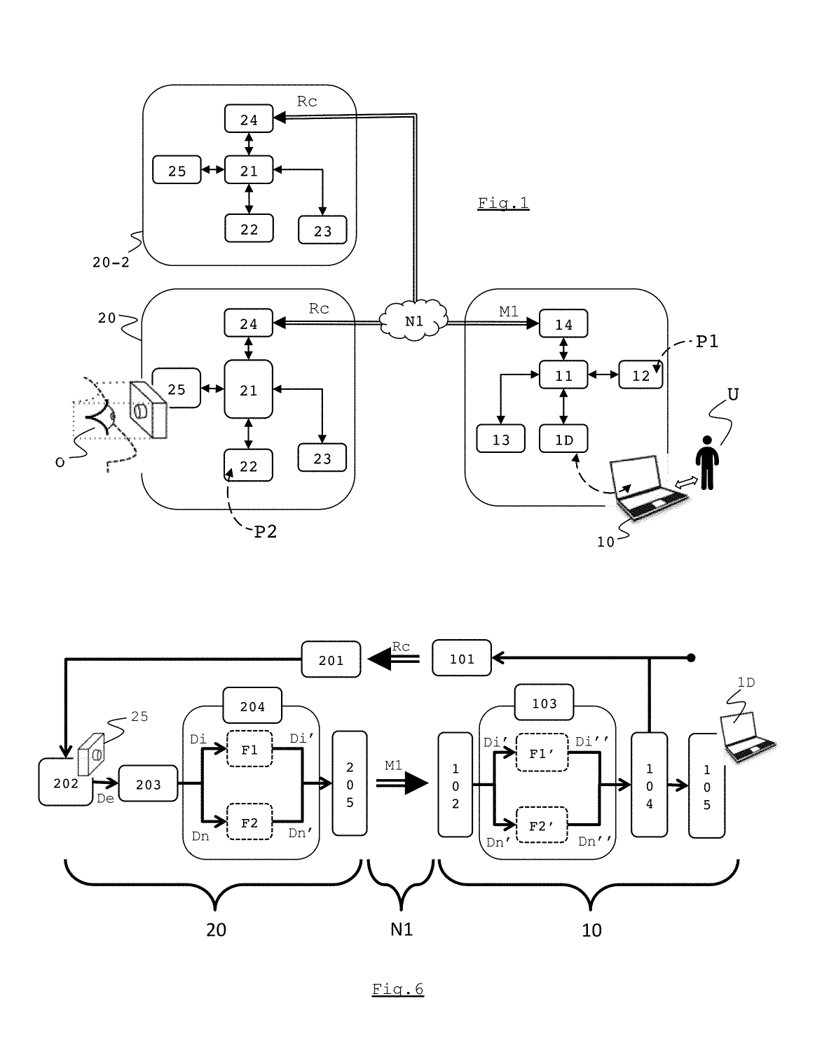

FIG. 1 shows a system according to the invention;

FIG. 2 shows a block diagram of a method according to the invention for interpreting a composite data stream;

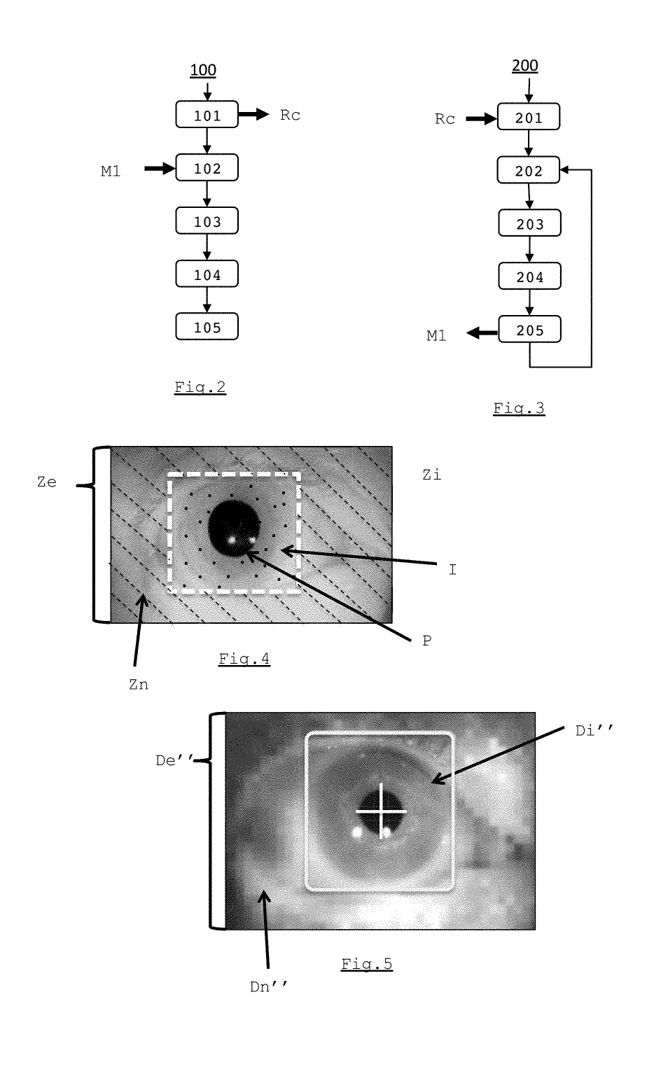

FIG. 3 shows a block diagram of the method according to the invention for sending a composite data stream;

FIG. 4 shows a study scene captured by a system according to the invention;

FIG. 5 shows a retrieval of a composite image by a system according to the invention;

FIG. 6 shows a block diagram implemented by a system according to the invention.

By way of preferred, but non-limiting application, the invention will be described through an application relative to generating, sending and interpreting a composite video stream, as produced during an examination of the vestibular system of a human or animal, or more generally of a study subject.

A study scene according to the invention, in connection with the examination of the vestibular system, includes the region of an eye O of a patient, which we will call study zone Ze, as described by FIG. 4. The study zone Ze is the zone observed by capture means 25 located, by way of non-limiting example, across from said eye O. The study zone Ze includes a zone of interest Zi including the pupil P and the iris I of said eye O represented by dotted lines in connection with FIG. 4, an environmental zone Zn corresponding to the study zone Ze taken from the zone of interest Zi represented by crosshatching in connection with FIG. 4.

FIG. 1 makes it possible to show an example system according to the invention. Such a system consists of an electronic device 10 cooperating with one or several communicating electronic objects 20, 20-2 via a wired or wireless communication link N1. Such electronic objects 20, 20-2, referenced 20 in the rest of the document for simplification reasons, include capture means 25 cooperating with a processing unit 21. Such capture means 25 may consist of a matricial sensor or a single infrared sensor. The processing unit 11 is responsible for collecting the data delivered by the capture means 25 and encoding it before sending it to the electronic device 10 via communication means 24. The processing unit 21 then advantageously includes one or several microcontrollers or processors cooperating by coupling and/or by wired bus, shown by double arrows in FIG. 1, with the capture means 25. The capture means 25 may, additionally or alternatively, deliver other study information, for example in connection with the trajectory and/or the movements made by the patient. Such means 25 may then, for example, include an inclinometer, a gyroscope or an accelerometer, or more generally any sensor making it possible to determine a movement, such as, but not limited to, a movement of a patient's head. More generally, the capture means 25 can measure one or several physical properties in connection with the study subject. The capture means 25 produce a digital representation R(t) of the study zone Ze. The latter is recorded in the storage means 22, 23 cooperating by coupling and/or wired bus with the processing unit 21. Such storage means 22, 23 may consist of a data memory 23 arranged to record study data De characterizing the digital representation R(t) of the study zone Ze.

The storage means 22, 23 may further consist of a program memory 22 including the instructions of a computer program product P2. Said program instructions P2 are arranged such that their execution by the processing unit 21 of the object 20 causes the implementation of a method for generating and sending a composite data stream. Such storage means 22, 23 may, as an optional alternative, constitute only one same physical entity.

According to one preferred embodiment, the recording of a time datum t characterizing an acquisition period may be done jointly with that of the digital representation R(t). The latter, and therefore the capture done by the capture means 25, is thus time stamped.

As a non-limiting example, the capture means 25 may include a matricial sensor, such as an infrared camera. The digital representation R(t) delivered by such a sensor consists of a pixel table encoding a shade of gray for each of them. The capture means 25 may include or be associated with a light-emitting diode emitting in the infrared, not shown in FIG. 1, to allow adequate lighting for the acquisition of data by said capture means 25.

As previously mentioned, although FIG. 1 only explicitly describes a single piece of capture equipment 25 in the form of a camera, other identical or additional capture means may further be connected directly or indirectly to the processing unit 21. A system according to the invention may then include, by way of non-limiting examples, two cameras respectively capturing a scene of interest corresponding to the left eye of a patient and a second scene of interest corresponding to the right eye of said patient. Such a system may further include an angular sensor of the gyroscope type to provide the angular position of the patient's head in a given coordinate system and thus to be able to determine, by way of non-limiting example, the direction of rotation of the patient's head or body during an examination. A digital representation R(t) delivered by such an angular sensor could consist of an acceleration vector along several reference axes.

To be able to cooperate with an electronic device 10, as described in connection with FIG. 1, said electronic object 20 also includes communication means 24, in the form of a modulator-demodulator allowing an electronic object 20 to communicate through a communication network N1, for example of the Bluetooth or Wi-Fi type. Alternatively, the communication means 25 may further consist of a USB (Universal Serial Bus) port in order to implement a wired-type link N1.

Advantageously, such an electronic object 20 may have a battery, or more generally any internal electrical source, or be connected to the electric grid so as to draw sufficient electricity necessary for its operation therefrom. The use of an internal battery electrical source will be favored so as not to hinder the mobility of the object by the presence of a power cable.

As previously mentioned, an electronic object 20 according to the invention may, as non-limiting examples, consist of a mask, glasses or helmet positioned on the head of a patient and including capture means 25. Unlike the known solutions, it further includes a processing unit 21, storage means 22, 23, and communication means 24. Such an object 20 no longer requires wired connector technology to cooperate with a third-party device.

In this respect, FIG. 1 further describes such a third-party electronic device, such as a personal computer 10 or a touch-sensitive tablet, for example. Like an electronic object 20, said device 10 includes a processing unit 11, for example in the form of one or several microcontrollers or processors cooperating with storage means 12, 13, in the form of a program memory 12 and a data memory 13, said data memory 12 and program memory 13 being able to be separated or optionally to form a same physical entity. The electronic device 10 may be suitable for interpreting a composite data stream by loading a computer program product P1 according to the invention into the program memory 12. Said electronic device 10, thus adapted, becomes capable of receiving, interpreting and exploiting a composite data stream in the form of one or several incoming messages M1 conveyed by a communication network N1, advantageously exploiting a wireless communication protocol of the Wi-Fi or Bluetooth type. We note that the invention does not exclude a wired communication mode as such, for example of the USB type, so as no longer to be penalized by the bandwidth limits imposed by such a link. The electronic device 10 thus includes communication means 14 arranged to provide such communication, by receiving messages M1 previously encoded by an electronic object 20. The storage means 12, 13 and the communication means 14 advantageously cooperate with the processing unit 11 by one or several communication buses, shown by double arrows in FIG. 1.

In one preferred example of the invention, which is not limiting, the data memory 13 may be arranged to record the content of the messages M1 received by the communication means 14.

An electronic device 10 further advantageously includes a man-machine input and/or output interface 1D cooperating with the processing unit 11. Said interface 1D makes it possible to output, for a user U of said device 10, for example the content of the data transmitted by the electronic object 20 or produced by the processing unit 11 of said device 10. Said man-machine input and/or output interface 1D may further make it possible to translate a gesture or voice command from said user U into input data C able to be interpreted by the processing unit 11 of said device 10. Such an interface 1D may for example consist of a touch-sensitive screen or assume the form of any other means allowing a user U of the device 10 to interact with said electronic device 10. Alternatively, the electronic device 10 may include two separate man-machine interfaces to translate inputs from the user U and to output graphic and/or audio content for the latter. For example and non-limitingly, such an input interface may consist of a keyboard or microphone and such an output interface may consist of a monitor or a speaker.

To illustrate the contribution of the invention, let us study a case according to which a capture means 25, for example of the image matricial sensor type, captures the eyeball of a patient. The zone of interest Zi is the made up of the pupil P and the iris I of an eye O and the environmental zone Zn by the eyeball and eyelid of the patient, as shown in connection with FIG. 4.

FIG. 3 describes a block diagram according to the invention of a method 200 for generating a composite data stream.

A method 200 according to the invention and implemented by a processing unit 21 of an electronic object 20, as described in connection with FIG. 1, includes a first step 202 for triggering the capture of a study zone or scene Ze. Said step 202 consists of producing a digital representation R(t₁) of the study zone Ze in connection with a current period t₁. The study data De characterizing the computer transcription of the digital representation R(t₁) is stored within storage means 22, 23, for example in the form of a structure of the integer array type, each field respectively being associated with different pixels and describing a gray level of said associated pixel, for example a zero-value describing a low gray level, such as black, and a value equal to 256 describing a high gray level, such as white. Such a structure may further, and as a non-limiting example, record one or several attributes related to the capture of the study scene. For example, such an attribute may record the acquisition period t₁ and an identifier Idc characterizing the capture means 25.

The method 200 also includes a step 203 for discriminating from among the study data De, data of interest Di and environmental data Dn. Such a step 203 consists of carrying out a function or discrimination processing operation making it possible to identify and isolate data of interest Di according to a predefined criterion. For example, one seeks to separate the data Di in connection with the pupil P and the iris I of the studied eye in light of the data Dn in connection with the rest of the eyeball. Such a discrimination function may consist of analyzing the digital representation, i.e., the study data De, from the captured study zone, for example in the form of a table of pixels including the light intensities in gray level of each pixel, and seeking, using the known image analysis methods, the location of the pupil of the eye. Such image analysis methods can, by way of non-limiting example, consist first of performing thresholding of the study data De in order to obtain a binary digital representation of the image, i.e., including only two values. Such thresholding may for example consist of replacing the value of the gray level with a zero value of all of the pixels having a gray level below a predetermined value and replacing the gray level value with a maximum gray level value of all of the pixels having a gray level greater than or equal to said predetermined value. By way of non-limiting example, for an image with 256 gray levels, the predetermined value can be set at 125. Another thresholding technique may consist of calculating the histogram of the image, i.e., determining the distribution of the light intensities of the pixels of the image, then modifying the general intensity of the image to increase the shades of gray thereof in order to increase the contrasts. After thresholding, such a method may carry out a so-called cleaning step. The cleaning may, by way of example, consist of a morphological analysis of the image from predetermined rules. For example, a pupil of an eye is generally circular. Such cleaning may then consist of excluding, from the analysis of the image, all of the areas with noncircular shapes, for example oblong shapes being able to characterize a makeup zone on top of the eyelid of the eye. Lastly, such an image analysis method may include a computing step for roughly estimating the placement of the pupil. Such a step may, by way of example, consist of virtually isolating a group of pixels representing a disc and having a low gray level close to black, then determining the center of said disc, for example by computing its barycenter.

Once the location of the center of the pupil is determined, the discrimination function may provide for defining a geometric shape around said center, for example a square or a circle with determined sides or a determined radius, said geometric shape encompassing the pixels of the zone of interest, in the case at hand the pupil and the iris. By way of non-limiting example, said geometric shape may consist of a square with sides measuring 12 millimeters or 144 pixels. The invention then provides for discriminating the data of interest Di as being recorded in a second memory structure of the integer array type associated with only pixels captured by said geometric shape. Such a second data structure Di can be recorded, by way of non-limiting example, in the data memory 23 of said object 20.

The environmental data Dn may consist of the structure recording the study data De or, alternatively, result from a copy thereof for which the intensity values associated with the pixels of interest have been replaced by a predetermined value, for example 0.

According to one alternative of the invention, the storage means 22, 23 of the electronic object 20 include a recording arranged to store zone of interest parameters PI making it possible to discriminate the data of interest Di within study data De produced by the capture means 25 of the electronic object 20. Such parameters thus designate the data Di. By way of non-limiting example, such zone of interest parameters PI may correspond to a pair of coordinates X, Y designating a referring pixel, i.e., the data associated with said pixel when the latter are advantageously arranged in the form of a table in storage means 22, 23 of the object 20. Such zone of interest parameters PI further include a pair of values W, H characterizing a width and/or a length of a geometric figure, for example a square, delimiting a desired zone of interest. Step 203 for discriminating the data of interest Di from among the study data De and thus distinguishing them from the environmental data Dn then consists of extracting said zone of interest parameters PI and implementing a discrimination function according to the content of said zone of interest parameters PI.

According to one alternative of the invention, the data of interest Di may correspond to the study data set De.

The method 200 then includes a step 204 for encoding the data of interest Di according to a first encoding function F1 and the environmental data Dn according to a second encoding function F2. By way of non-limiting examples, the encoding functions F1, F2 can respectively be based on encoding parameters E1, E2, characterizing an encoding standard, a desired image resolution, expressed in number of pixels, and/or irreversible compression parameters characterizing a pixel binning rate, etc. Thus, such first encoding parameters E1 can characterize an attribute translating an RLE (Run Length Encoding) standard, an attribute characterizing an image of 744×480 pixels, an attribute characterizing a zero-binning rate, an attribute requesting the transmission of all of the light intensities of the pixels. By way of non-limiting example, the second encoding parameters E2 encoding the environmental data Dn may conversely characterize an attribute translating a JPEG standard, an attribute characterizing an image of 320×240 pixels, an attribute characterizing a binning rate of 50%, an attribute characterizing the sending of the light intensities of only the pixels with an odd rank. The data of interest Di thus encoded, hereinafter referred to as Di′, may then be recorded in the form of a data table. The same is true for the encoded environmental data Dn, hereinafter referred to as Dn′. The data memory 23 may thus include a table of encoded environmental data Dn′ and a table of encoded data of interest Di′. We can see that, according to the content of the parameters E1 and E2, the encoded data of interest Di′ can thus be deteriorated less than the encoded environmental data Dn′.

Such encoding parameters E1, E2 can advantageously be recorded in the storage means 22, 23 of the electronic object 20, in the form of one or several recordings. Step 204 for encoding the data of interest Di according to a first encoding function F1 and the environmental data Dn according to a second encoding function F2 then consists of extracting, before encoding the data Di and Dn as such, said parameters E1, E2 and formulating, configuring or choosing the first and second encoding functions F1 and F2 from encoding parameters E1 and E2.

The method 200 then includes a step 205 for generating and triggering the sending, by the communication means 24 to an electronic device 10, of one or several messages M1 each including all or part of the data Di′ and/or Dn′ and a descriptor, for example in the form of a header.

A message descriptor M1 may for example include an identifier Idc of the capture means 25, a field including an identifier of the acquisition period t of the study data from which the data Di′ and/or Dn′ are derived, an attribute characterizing the type of study data set, an encoding attribute designating the encoding function of said sent data, an identifier of the object sending said message Ml, or even an identifier of the device receiving said message Ml. Such a descriptor may further include an attribute characterizing a coordinate system shared between the data of interest Di′ and environmental data Dn′ sent in a batch of messages Ml. Such a coordinate system may, for example, consist of the coordinates of a reference pixel present in the two digital representations of the zone of interest Zi and the environmental zone Zn, for example a pixel associated with a pupil center.

The sending of the data previously encoded Di′ and Dn′ may be translated in a plurality of messages M1 whose respective descriptors may further include a sequential indicator, to sequence said messages when they are received, or even redundancy, integrity and/or any encryption data.

The electronic device 10 receives, through its communication means 14, one or several messages NA1 during a step 102 of the method 100 for interpreting a composite data stream, one example of such a method 100 being described in connection with FIGS. 2 and 6.

Such a method 100 for interpreting a composite data stream is carried out by the processing unit 11 of said device 10. The method 100 includes a step 103 for decoding the content of said message Ml. Such a step 103 may consist of decoding and extracting, from the descriptor of said message M1, the attribute characterizing the type of study data sent and the encoding attribute designating the encoding function of the study data implemented by the electronic object 20 having sent said message M1. Step 103 next consists of decoding the study data sent according to a decoding function Fr, F2′. The choice and/or the configuration of such a decoding function may depend on said encoding attribute thus decoded or may be predetermined. Alternatively, such an attribute may further include an encoding parameter E1 or E2 for an encoding function F1 or F2 implemented by the object 20.

By way of non-limiting example, the study data De′ conveyed in the message M1 may consist of encoded data of interest Di′ and/or encoded environmental data Dn′. Step 103 then consists of carrying out a first decoding function F1′ to decode the data of interest Di′ and a second decoding function F2′ to decode said environmental data Dn′. The storage means 12, 13 of the device 10 advantageously include data structures, for example in the form of one or several integer arrays, to store the data of interest Di″ and the environmental data Dn″ thus decoded. Such tables may thus, for example, record light intensities and/or gray levels of pixels of an image that the device 10 may recompose. Said arrays are then digital representations similar to those of the zone of interest Zi and the environmental zone Zn previously captured by the electronic object 20. Indeed, as we saw previously, the study data De can have been encoded and sent according to an irreversible compression factor implemented according to an encoding function F1, F2, for example encoding and sending only the light intensities of only the odd pixels. The decoding functions Fr, F2′ must then, by way of non-limiting example, interpolate the study data De′ thus received while recording, in the odd ranks of the integer arrays, the value of the light intensities of the lower even ranks. Any other interpolation function could, alternatively, be implemented.

The method 100 includes a step 104 for jointly explaining said data of interest Di″ and environmental data Dn″ thus decoded.

Such an exploitation may, by way of non-limiting example, consist of producing time stamped recordings dedicated to archiving in the data memory 13 to create a history of the decoded data of interest Di″ and environmental data Dn″. Such storage may, by way of non-limiting example, be arranged to store the acquisition period t of the study data from which are derived the data Di′ and/or Dn′ previously extracted from the message M1 and the integer tables associated with the decoded data of interest Di″ and environmental data Dn″. Such archiving may be exploited for computation purposes or for the generation of new data. By way of non-limiting example, when the study data relate to acceleration vectors delivered by a gyroscope, such archiving may make it possible to reproduce the trajectory and speed of the eyeball during an exam. Alternatively or additionally, the study data may be produced from a matricial image sensor of the eyeball. To determine the trajectory of said eyeball, the processing unit 11 of the device 10 may determine the location of the pupil center according to known image analysis methods previously described, from data of interest Di″ extracted from several recordings dedicated to archiving. The data memory 13 then includes a trajectory output data structure including several recordings arranged to record trajectory output data Dr including said pupil center locations and the associated acquisition period.

By way of preferred, but non-limiting example, a joint exploitation of the decoded data of interest Di″ and decoded environmental data Dn″ and derived from a same acquisition by the capture means 25 of the object 20 may consist of re-composing an image of the study scene Ze intended to be output by an output interface 1D of the electronic device 10 or cooperating with the latter, from said data Di″ and Dn″. The data memory 13 then includes an image output structure, for example in the form of an integer array, to record image output data Dr necessary for the generation of such an image. Said image output data Dr consist of data of interest Di″ and environmental data Dn″ previously decoded and derived from a same acquisition. Step 104 may then consist of recording, in the output table, the content of the decoded environmental data Dn″, then virtually superimposing the data of interest Di″ on the environmental data Dn″. Such a superposition may consist of determining the location of the data of interest Di″ relative to the environmental data Dn″ from the shared coordinate system extracted from the message M1 previously received. The value of the light intensities of the shared pixels are then replaced by those of the data of interest Di″.

The output data Dr thus created may be exploited later or output on an output interface 1D of the electronic device 10.

To that end and according to the example described in connection with FIG. 2 or 6, the method 100 further includes a step 105 for triggering the output, by a man-machine output interface 1D of the electronic device 10, of all or part of the output data Dr previously generated. As a non-limiting example, such an output may consist of displaying the trajectory output data Dr in the form of a graph showing the location of the pupil center as a function of time.

Alternatively, such an output may consist of displaying the image output data Dr in the form of an image showing a reconstruction of the study zone Ze, as shown in connection with FIG. 5. As one can see in FIG. 5, the curve of the eyelid of the observed eye is output in the form of a staircase of pixels in the part located outside the white square and corresponding to the decoded environmental data Dn″. On the contrary, in the zone inside the white square and corresponding to the decoded data of interest Di″, the curve of the eyelid is output very smoothly. One can indeed see two different image resolutions.

Some healthcare staff may be bothered by an image including two different resolutions, as shown in FIG. 5. The practitioner may consider that too much information is lost between the capture of the eye by the electronic object 20 and its output by the device 10. To facilitate practitioner adhesion to a composite image and focusing of his gaze on the output zone of interest Zi″, the invention provides for inserting a line of demarcation therein, as shown by the white square in FIG. 5. Such a line of demarcation may, as a non-limiting example, be inserted in the image retrieval data Dr by replacing the value of the pixels of the bordering environmental data Dn″ with the data of interest Di″ by a value equal to 256, describing a high gray level, such as white. The detection of bordering pixels may be done using known image processing methods.

Furthermore, the invention may provide for superimposing, on the output of the composite image, additional metadata, for example, but not limited to, the acquisition period t of the output image and the location of the pupil center shown by a cross in connection with FIG. 5.

According to the invention, the electronic object 20 may include a plurality of capture means 25, for example, but not limited to, a matricial image sensor and a gyroscope, respectively producing first study data De1 relative to the eyeball and second study data De2 relative to the rotation direction of a patient's head, said first and second study data De1, De2 being captured during the same acquisition period t₁. The method 200 for sending a composite data stream and the method 100 for interpreting such a stream, both previously described, are then respectively carried out for the processing unit 21 of the object 20 from said first and second study data De1 and De2 and by the processing unit 11 of the device 10 from decoded first and second study data De1″ and De2″. The encoding functions F1, F2, . . . , Fx and decoding functions Fr, F2′, . . . , Fx′ associated with the first and second data of interest Di1, Di2 and/or environmental data Dn1, Dn2 respectively derived from the first and second study data De1 and De2 can advantageously be identical or different, depending on the nature of the exchanged data.

Step 104 of the method 100 for exploiting the decoded study data De1″ and De2″ may then consist of producing first and second output data Drl and Dr2 respectively from first and second decoded study data De1″ and De2″. Step 105 for triggering the output of all or some of the output data Drl, Dr2 via an output interface 1D may then consist of embedding output data Dr2 in the output data Drl. By way of non-limiting example, such an embedding may consist of inserting a representation of an arrow translating the direction of movement of the patient's head in the output of the composite image derived from the output data Drl.

According to one alternative of the invention, certain processing operations carried out by the processing unit 21 of the electronic object 20 may be delegated and therefore carried out in place of the electronic object 20, by the electronic device 10.

By way of non-limiting example, the electronic device 10 may inform the object 20 of the position of the zone of interest Zi during the following acquisition or indeed impose it as of the beginning of the implementation of the method 200. Said method 100 to that end includes a step 101, prior to the step 102 for receiving a message Ml, for generating a command request Rc1 including previously defined zone of interest parameters PI and designating data of interest Di within study data De produced by the capture means 25 of the electronic object 20 that the object 20 will have to discriminate. The method 200 for generating a composite data stream then includes a step 201 for receiving, via the communication means 24 of said object 20, said command request Rc1, then decoding and extracting said zone of interest parameters PI. The step 203 for discriminating, in the study data De, data of interest Di and environmental data Dn then consists of carrying out a discrimination function according to the content of said zone of interest parameters PI.

According to another alternative of the invention, step 101 for generating a command request Rc1 may further consist of generating a second command request Rc2 including encoding parameters for all or part of the study data De. As an example, such a request Rc1 may include first encoding parameters E1 and second encoding parameters E2 specific to the data of interest Di and environmental data Dn. Step 201 for receiving and decoding the command request of the method 200 carried out by the processing unit 21 of the electronic object 20 may then consist of extracting the content of said encoding parameters E1 and E2 of the second command request Rc2. Step 204 for encoding the data of interest Di according to a first encoding function F1 and the environmental data Dn according to a second encoding function F2 then consists of carrying out said first and second encoding functions F1 and F2 respectively according to the content of said first and second encoding parameters E1 and E2.

Alternatively, said first and second command requests Rc1 and Rc2 may consist of a single request Rc3 including zone of interest parameters PI and first and second encoding parameters E1, E2 specific to the data of interest Di and environmental data Dn.

According to one alternative of the invention, the electronic object 20 may send a video stream to the electronic device 10. The method 200 for generating a composite data stream is then carried out iteratively, for example, every 20 milliseconds or every 5 milliseconds.

Alternatively, the command requests Rc1 and/or Rc2 and/or Rc3 may include an attribute characterizing the capture frequency done by the capture means 25 and sending by the communication means 24 of the study data De. As an example, such a frequency may be equal to 50 Hz or 200 Hz.

1. A method for interpreting a composite data stream implemented by a processing unit of an electronic device cooperating with at least one communicating electronic object including capture means, said device including, aside from said processing unit, communication means providing a communication mode with said electronic object through a communication network, wherein:
the data stream includes study data previously encoded by the electronic object, said study data including data of interest encoded according to a first encoding function and environmental data encoded according to a second encoding function, said study data being produced beforehand by said capture means of said electronic object;
and wherein the method comprises:
a step for receiving, via the communication means, a message including said encoded study data;
a step for decoding said encoded study data, said step comprising decoding said encoded data of interest and environmental data respectively according to first and second determined decoding functions; and
a step for jointly exploiting said data of interest and environmental data thus decoded.

2. The method according to claim 1, wherein the message further includes a descriptor translating a coordinate system shared by the encoded data of interest and environmental data, the step for jointly exploiting said data comprising producing retrieval data from decoded data of interest and environmental data and said shared coordinate system.

3. The method according to claim 1, including a step prior to the step for receiving a message including encoded data of interest and environmental data, for developing and triggering the sending by the communication means to the electronic object of a command request able to be interpreted by said electronic object including a first encoding parameter and a second encoding parameter, said first and second parameters being different.

4. The method according to claim 1, including a step, prior to the step for receiving a message including encoded data of interest and environmental data, for developing and triggering the sending by the communication means to the electronic object of a command request able to be interpreted by said electronic object including a zone of interest parameter designating data of interest within study data produced by the capture means of the electronic object, said zone of interest parameter being able to be interpreted by the electronic object to discriminate the data of interest from the environmental data previously produced by said object.

5. The method according to claim 1, wherein:
the electronic device includes an output interface cooperating with the processing unit of said device;
the step for jointly exploiting said decoded data of interest and environmental data comprises producing output data;
said method includes a step after said step for jointly exploiting said decoded data of interest and environmental data, to trigger the output of said output data via the output interface.

6. The method according to claim 1, wherein the capture means include a matricial image sensor, the study data produced by said capture means comprising a digital representation of a scene captured by said matricial image sensor.

7. The method according to claim 6, wherein the scene captured by said matricial image sensor includes an eye of a study subject, and the data of interest are the digital representation of the iris and the pupil of said eye.

8. A computer readable medium encoded with program instructions which, when recorded in a program memory of an electronic device including the program memory, a processing unit, and communication means providing a determined communication mode, said program memory and said communication means cooperating with said processing unit, cause the implementation of a method for interpreting a composite data stream according to claim 1.

9. An electronic device including a processing unit, a program memory, a data memory, communication means cooperating with said processing unit, said device comprising, in the program memory, instructions for a computer program that implements the method for interpreting a composite data stream according to claim 1.

10. A method for sending a composite data stream implemented by a processing unit of a communicating electronic object cooperating with at least one electronic device, said electronic object further including capture means and communication means providing a communication mode with said electronic device through a communication network, said method comprising:
a step for triggering the production of study data by said capture means of said electronic object, said study data comprising a digital representation of a measured physical property;
a step for discriminating, in the study data, data of interest and environmental data according to a discrimination function;
a step for encoding the data of interest according to a first encoding function and environmental data according to a second encoding function; and
a step for generating and triggering the sending, by the communication means to the electronic device, of a message including all or some of said encoded data of interest and/or environmental data.

11. The method according to claim 10, including a step before the step for encoding the data of interest according to a first encoding function and the environmental data according to a second encoding function, for receiving, via the communication means, a command request sent from the electronic device including a first encoding parameter and a second encoding parameter, and extracting said parameters, said first and second encoding functions respectively being implemented according to the content of the first and second encoding parameters.

12. The method according to claim 10, including a step prior to the step for discriminating, in the study data, the data of interest and the environmental data according to a discrimination function, to receive, via the communication means, a command request sent from the electronic device including a zone of interest parameter designating data of interest and to extract said parameter, said discrimination function being implemented according to the content of said zone of interest parameter.

13. The method according to claim 10, wherein:
the electronic object further includes storage means including a zone of interest parameter designating data of interest from among the study data;
said method includes a step prior to the step for discriminating, in the study data, data of interest and environmental data, to extract, from said storage means, said zone of interest parameter designating data of interest and carrying out a discrimination function according to the content of said parameter.

14. The method according to claim 10, wherein:
the electronic object further includes storage means including first and second encoding parameters;
the step for encoding the data of interest according to a first encoding function and the environmental data according to a second encoding function further comprise extracting the content of said encoding parameters, said first and second encoding functions respectively being carried out according to said content of the first and second encoding parameters.

15. The method according to claim 10, wherein the capture means of the electronic object include a matricial image sensor, the study data produced by said sensor being the digital representation of a scene captured by said matricial sensor.

16. A computer readable medium encoded with program instructions which, when recorded in a program memory of a communicating electronic object including said program memory, a processing unit, communication means providing a determined communication mode, and capture means, said program memory, said communication means, and said capture means cooperating with said processing unit, cause the implementation of a method for sending a composite data stream, according to claim 10.

17. A communicating electronic object including a processing unit, a program memory, a data memory, communication means, capture means cooperating with said processing unit, said object comprising, in the program memory, instructions for a computer program that implements the method for sending a composite data stream according to claim 10.

18. The communicating electronic object according to claim 17, wherein:
the capture means include a first capture means and a second capture means to respectively produce first and second study data; and
the processing unit then carries out the method for sending a composite data stream, for each of said first and second capture means.

19. A system including an electronic device according to claim 9, and at least one communicating electronic object including a processing unit, a program memory, a data memory, communication means, and capture means cooperating with said processing unit.

20. The system according to claim 19, comprising a videonystagmoscopy system, for which the electronic device comprises a personal computer and the electronic object comprises a videonystagmoscopy mask including at least one matricial image sensor.

21. A data processing method, including:
a step for triggering a production of study data by capture means of a communicating electronic object of a system that further includes an electronic device, said step being carried out by a processing unit of said object;
a step for discriminating, in the study data, data of interest and environmental data via said electronic object;
a step for encoding, via said object, the data of interest according to a first encoding function and the environmental data according to a second encoding function;
a step for generating, via said object, a message including said encoded data of interest and said encoded environmental data and triggering the sending to the electronic device the system; and
a step for receiving, via the device, said message and decoding it;
a step for jointly exploiting said decoded data of interest and environmental data via the device.

22. The data processing method according to claim 21, including:
a step for generating and triggering the sending, through a communication network to the communicating electronic object, a command request including encoding parameters and a zone of interest parameter implemented by the processing unit of an electronic device;
the step for receiving, via the communicating electronic object, said command request and extracting said parameters;
the discriminating step being carried out according to the contents of the zone of interest parameter previously extracted;
the step for respectively encoding the data of interest and the environmental data being carried out by said object according to the content of said encoding parameters previously extracted.
